(19) United States
(12) Patent Application Publication
(10) Pub. No.: US 2008/0090237 A1
(43) Pub. Date: Apr. 17, 2008

(54) Methods and compositions for comparing chromosomal copy number between genomic samples

(76) Inventors: Zohar Yakhini, Ramat HaSharon (IL); Bo Curry, Redwood City, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/581,134

(22) Filed: Oct. 13, 2006

(51) Int. Cl. C12Q 1/68; G06F 19/00

(52) U.S. Cl. 435/6; 702/20

(57) ABSTRACT

Methods and compositions for detecting copy number variations between nucleic acid samples are provided. Also provided are kits for practicing methods in accordance with the invention.

### BACKGROUND OF THE INVENTION

Studying differences in gene dosage and DNA copy number among cell populations will lead to an improved understanding of human disease conditions and possibly to the development of accurate diagnostic assays based on DNA copy number variation. Many genomic and genetic studies are directed to the identification of differences in gene dosage or expression among cell populations for the study and detection of disease. For example, many malignancies involve the gain or loss of DNA sequences (alterations in copy number) and sometimes of entire chromosomes, resulting in activation of oncogenes or inactivation of tumor suppressor genes. Furthermore, alterations in genetic copy number are associated with a variety of non-neoplastic diseases and developmental disorders such as trisomy 21. Thus, identification of the genetic and epigenetic events in normal and abnormal cell types and tissues, as well as those leading to neoplastic transformation and subsequent disease progression, can facilitate efforts to define the biological basis for disease and development, develop predictors of disease outcomes, improve prognosis of therapeutic response, and permit earlier disease detection.

### SUMMARY OF THE INVENTION

Methods and compositions for detecting copy number variations between nucleic acid samples are provided. Also provided are kits for practicing methods in accordance with the invention.

### BRIEF DESCRIPTION OF THE FIGURES

FIG. 1 schematically illustrates exemplary detection nucleic acid pairs that find use in the copy number variation analyses of the present invention.

### DEFINITIONS

The term “nucleic acid” and “polynucleotide” are used interchangeably herein to describe a polymer of any length, e.g., greater than about 5 bases, greater than about 100 bases, greater than about 500 bases, greater than 1000 bases, usually up to about 10,000 or more bases composed of nucleotides, e.g., deoxyribonucleotides or ribonucleotides, or other compounds (e.g., PNA as described in U.S. Pat. No. 5,948,902 and the references cited therein), produced either synthetically or in vivo, which can hybridize with naturally occurring nucleic acids in a sequence specific manner analogous to that of two naturally occurring nucleic acids, e.g., can participate in Watson-Crick base pairing interactions. Naturally occurring nucleotides include guanine, cytosine, adenine and thymine (G, C, A and T, respectively).

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 5 to 100 nucleotides and up to 200 nucleotides in length. Oligonucleotides are usually synthetic and, in many embodiments, are fewer than 70 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “labeled population of nucleic acids” refers to a mixture of nucleic acids that are detectably labeled, e.g., fluorescently labeled or mass tagged, such that the presence of the nucleic acids can be detected by assessing the presence of the label. A labeled population of nucleic acids is “made from” a “genomic composition” or a “sample composition”, the composition is usually employed as template for making the population of nucleic acids.

The term “array” encompasses the term “microarray” and refers to an ordered array presented for binding to nucleic acids and the like.

The term “stringent assay conditions” as used herein refers to conditions that are compatible with producing binding pairs of nucleic acids, e.g., probes and targets, of sufficient complementarity to provide for the desired level of specificity in the assay while being incompatible with the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5-SSC, and 1% SDS at 42° C., or hybridization in a buffer comprising 5-SSC and 1% SDS at 65° C., both with a wash of 0.2-SSC and 0.1% SDS at 65° C. Exemplary stringent hybridization conditions can also include a hybridization in a buffer of 40% formamide, 1 M NaCl, and 1% SDS at 37° C., and a wash in 1-SSC at 45° C. Alternatively, hybridization to filter-bound DNA in 0.5 M NaHPO₄, 7% sodium dodecyl sulfate (SDS), 1 mM EDTA at 65° C., and washing in 0.1-SSC/0.1% SDS at 68° C. can be employed. Yet additional stringent hybridization conditions include hybridization at 60° C. or higher and 3-SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily-recognize-that alternative but comparable hybridization and wash conditions can be utilized to provide conditions of similar stringency.

In certain embodiments, the stringency of the wash conditions determines whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2-SSC at a temperature of at least about 50° C. or about 55° C. to about 60° C for about 15 to about 20 minutes; or, the hybridization complex is washed twice with a solution with a salt concentration of about 2-SSC containing 0.1% SDS at room temperature for 15 minutes and then washed twice by 0.1-SSC containing 0.1% SDS at 68° C. for 15 minutes; or, equivalent conditions. Stringent conditions for washing can also be, e.g., 0.2-SSC/0.1% SDS at 42° C. In instances wherein the nucleic acid molecules are deoxyoligonucleotides (“oligos”), stringent conditions can include washing in 6-SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5-SSC and 0.1-SSC at room temperature.

Stringent hybridization conditions may also include a “prehybridization” of aqueous phase nucleic acids with complexity-reducing nucleic acids to suppress repetitive sequences and reduce the complexity of the sample prior to hybridization. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable. To be specific, an array of surface-bound polynucleotides, as is commonly known in the art and described below, is not a mixture of surface-bound polynucleotides because the species of surface-bound polynucleotides are spatially distinct and the array is addressable.

The term “mass tag”, as used herein, means any chemical moiety (i) having a fixed mass, (ii) affixable to a nucleic acid, and (iii) whose mass is determinable using mass spectrometry. Mass tags include, for example, chemical moieties such as small organic molecules. In certain embodiments, mass tags have masses which range from about 100 Da to about 10,000 Da.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well known in the art and include, for example, ion-exchange chromatography, affinity chromatography and sedimentation according to density. Generally, a substance is purified when it exists in a sample in an amount, relative to other components of the sample that is not found naturally.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent. Additionally, the “binding characteristic” of a target to a probe means the result of measuring the amount of target associated with a probe after contacting the target (or target sample) to a probe.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

By a detection nucleic acid “corresponding to”, being “designed for” or being “specific for” a certain nucleic acid region (e.g., a genomic locus) is meant that the detection nucleic acid binds to the nucleic acid region under stringent hybridization conditions (e.g., as described above). In certain embodiments, a detection nucleic acid specific for a nucleic region contains a domain that is complementary to all or a portion (e.g., about 75% or more, such as about 90% or more including about 95% or more) of a nucleic acid region of interest (e.g., promotes Watson-Crick binding to the nucleic acid region of interest).

By “detection nucleic acid pair” or “matched pair of detection nucleic acids” is meant a pair of detection nucleic acids whose cognate binding sites are directly adjacent in the nucleic acid region of interest. By “directly adjacent” is meant that the pair of detection nucleic acids bind to sequences which, as found in the nucleic acid locus, are contiguous and that the binding of the detection pair is non-overlapping, with the 3′-most base of a first of the pair directly preceding the 5′-most base of a second of the pair. In certain embodiments, the members of a matched detection pair are described as binding to “flanking regions” of the nucleic acid locus of interest.

### DETAILED DESCRIPTION

As summarized above, methods and compositions for comparing the copy number of a locus of interest in nucleic acid samples are provided. Certain embodiments of the invention include comparing copy number at one or more loci between several chromosomal samples. Certain embodiments of the invention provide a first and second matched pair of detection nucleic acids, where each matched pair contains nucleic acids that hybridize to a genomic locus of interest at adjacent locations such that the locus serves to position each pair to be covalently joined (e.g., by ligation or other techniques). The first matched pair is designed to differ in at least one physical parameter from the second matched pair (e.g., by mass, length, label, etc.) such that, when covalently joined, the pairs can be differentiated using any of a number of detection methods.

In certain embodiments of the methods of the invention, the first matched pair of detection nucleic acids is hybridized to a reference sample and the second matched pair of detection nucleic acids is hybridized to a test sample. After hybridization, a ligating agent is contacted to each of the samples, which covalently joins detection primer pairs bound to the locus of interest, and the covalently joined detection primers from the reference and test samples are detected. A comparison of the copy number of the locus of interest between the test and references sample can then be determined. In certain embodiments, multiple loci of interest can be analyzed simultaneously by employing multiple matched pair sets of detection nucleic acids, where each matched pair set is directed to a different locus and each matched pair is distinguishable (i.e., by a physical parameter) from any other matched pair after covalent attachment. Such embodiments are sometimes called multiplex embodiments. Also provided are kits that include the subject nucleic acids.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, methods and materials according to certain embodiemnts are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

As summarized above, the subject invention provides matched pairs of detection nucleic acids specific for a nucleic acid region (e.g., a genomic locus) of interest and methods for using the same. In further describing the present invention, representative methods of using the subject detection pairs of nucleic acids to compare copy number of a genetic locus between two or more samples are described in greater detail. Embodiments of applications in which the subject invention may find use as well as kits for use in practicing methods in accordance with the invention, will also be described.

### Methods of Comparing Chromosomal Copy Number Between Genomic Samples

Aspects of the present invention provide methods of comparing the copy number of one or more nucleic acid regions (or loci) in two or more nucleic acid samples. In certain embodiments, the methods include comparing the copy number of one or more nucleic acid loci between a first nucleic acid sample (e.g., a reference sample) and a second sample (e.g., a test sample).

In certain embodiments, the nucleic acid samples of interest include, but are not limited to, genomic samples, cDNA samples, RNA samples (e.g., mRNA), synthetic nucleic acid samples, etc. For the purposes of description below, the samples described below will be genomic samples. However, this is not meant as a limitation of the invention as the invention is applicable to comparing loci between samples of varying types of nucleic acids.

As noted above, in certain embodiments, the nucleic acid sample is a genomic sample. In certain embodiments, the genomic sample is referred to as a genomic source. By “genomic source” is meant the initial nucleic acids that are used as the original nucleic acid source from which the solution phase nucleic acids employed in a given assay are produced. A genomic source may be prepared using any convenient protocol. In certain embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. However, in certain embodiments, the genomic source may comprise a portion of the genome, e.g., one or more specific chromosomes or regions thereof, such as PCR amplified regions produced with pairs of specific primers, or extrachromosomal elements such as mitochondria, viral particles, plasmids, or double minute chromosome fragments.

A given initial genomic source may be prepared from a subject, for example a plant or an animal. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 Mb, such that they may be about 1 Mb or smaller, e.g., less than about 500 kb, etc.

In certain embodiments, the subject from which a genomic source is obtained is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in certain embodiments are human or mouse subjects. In certain embodiments, the genomic source derived from a subject is complex, as the genome of a subject can contain at least about 1×10⁸ base pairs, including at least about 1×10⁹ base pairs, e.g., about 3×10⁹ base pairs.

In certain embodiments, the subject methods include obtaining a first genomic sample (i.e., a reference sample) and a second genomic sample (i.e., a test sample). In certain embodiments, the reference sample may contain genomic material from any cell of an organism with a genome, e.g., yeast, plants and animals, such as fish, birds, reptiles, amphibians and mammals. In certain embodiments, the reference sample is obtained from a subject or a tissue which is normal, e.g., known not to have a disease, condition, or other property that is being assessed in a test sample. In certain other embodiments, a reference sample is obtained form a subject or a tissue which is not normal, e.g., known to have a disease, condition of other property that is being assessed in the test sample. In still other embodiments, both normal and not normal reference samples are used, where in certain embodiments the not normal sample is a positive control for the test sample in the assay. In certain embodiments, reference samples containing genomic material from mice, rabbits, primates, or humans, etc, can be made and used. Other cells that may be used as a source of genomic material for use as reference samples include: monkey kidney cells (COS cells), human embryonic kidney cells (HEK-293, Graham et al. J. Gen Virol. 36:59 (1977)); baby hamster kidney cells (BHK, ATCC CCL 10); Chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); and mouse L cells (ATCC CCL-1). Additional cells (e.g. human lymphocytes) and cell lines will become apparent to those of ordinary skill in the art, and a wide variety of cell lines are available from the American Type Culture Collection (ATCC), 10801 University Boulevard, Manassas, Va. 20110-2209.

In certain embodiments, the initial genomic source may be fragmented in the generation protocol, as desired, to produce a fragmented genomic source. In certain embodiments, a fragmented genomic source contains genomic molecules having an average size range of about 10 kilobases (kb) or less, such as about 5 kb or less, and including about 1 kb or less. Genomic sample fragmentation may be achieved using any convenient protocol, including but not limited to: mechanical protocols, e.g., sonication, shearing, etc., chemical protocols, e.g., enzyme digestion, etc.

In certain embodiments, the genomic source is prepared as a suspension of metaphase chromosomes (Carrano et al., Pvoc. Natl. Acad. Sci USA 76:1382, 1979; Langlois et al., Pvoc. NatL. Acad. Sci USA 79:7876, 1982; and Speicher et al., Nature Genetics 12:368, 1996). In certain embodiments, a genomic source obtained as metaphase chromosomes will not be fragmented prior to hybridization with the subject detection nucleic acids. In certain embodiments in which the genomic sample contains relatively large nucleic acid molecules (e.g., such as non-fragmented chromosomes), hybridization can be performed using nucleic acids (e.g., detection pair nucleic acids) that are able to strand-invade DNA molecules under conditions where the DNA is in a native or semi-denatured state. For example, in order to promote strand invasion of a large chromosomal DNA molecule, elevated temperatures and duplex destabilizing buffer may be used. Examples of such hybridization conditions can be found in: Sambrook et al., Molecular Cloning: A Laboratory Manual, Cold Spring Sep. 23, 2004 Harbor Press, New York, 1989 and Ausubel et al., Current Protocols in Molecular Biology, Greene Publishing Associates, New York, 1996. In certain embodiments in which the genomic sample contains smaller nucleic acids (e.g., small chromosomes or chromosome fragments, e.g., less than 5 kb in length), denaturing conditions can be used.

In certain embodiments, the test and/or reference sample(s) have non-reduced complexity as compared to the initial genomic sample. A non-reduced complexity genomic sample is one that is produced in a manner designed to not reduce its complexity. A genomic sample is considered to be a non-reduced complexity product composition as compared to the initial nucleic acid source (e.g., genomic source) from which it is prepared if there is a high probability that a sequence of specific length randomly chosen from the sequence of the initial genomic source is present in the product composition, either in a single nucleic acid member of the product or in a “concatomer” of two different nucleic acid members of the product (i.e., in a virtual molecule produced by joining two different members to produce a single molecule). A more detailed description of non-reduced complexity target compositions is presented in (Agilent application 10031482-1), which is incorporated herein by reference.

A non-reduced complexity genomic sample can be readily identified using a number of different protocols. One convenient protocol for determining whether a given collection of nucleic acids is a non-reduced complexity collection of nucleic acids is to screen the collection using a genome wide array of features for the initial, e.g., genomic source of interest. Thus, one can tell whether a given genomic sample has non-reduced complexity with respect to its initial genomic source by assaying the composition with a genome wide array for the genomic source. The genome wide array of the genomic source for this purpose is an array of features in which the collection of features of the array used to test the sample is made up of sequences uniformly and independently randomly chosen from the initial genomic source. As such, sequences of a particular length independently chosen randomly from the initial genomic source that uniformly sample the initial genomic source are present in the collection of features on the array. By uniformly is meant that no bias is present in the selection of sequences from the initial genomic source. In such a genome wide assay of sample, a non-reduced complexity sample is one in which substantially all of the array features on the array specifically hybridize to nucleic acids present in the sample, where by substantially all is meant at least about 10%, for example at least about 25%, including at least about 50%, such as at least about 60, 70, 75, 80, 85, 90 or 95% or more.

As such, according to the above guidelines, a sample is considered to be of non-reduced complexity as compared to its genomic source if its complexity is at least about 10%, for example at least about 25%, including at least about 50%, such as at least about 60, 70, 75, 80, 85, 90 or 95% or more of the complexity of the genomic source.

In certain other embodiments, the test and/or reference genomic sample(s) may be of reduced complexity as compared to the initial genomic source. By reduced complexity is meant that the complexity of the target composition is at least about 20-fold less, such as at least about 25-fold less, at least about 50-fold less, at least about 75-fold less, at least about 90-fold less, at least about 95-fold less complex, than the complexity of the initial genomic source in terms of total numbers of sequences found in the test and/or reference samples as compared to the initial genomic source. Non-limiting examples of methods for reducing the complexity of a nucleic acid sample include subtractive hybridization, positive selection, size-fractionation, sequence specific amplification (e.g., PCR or linear amplification methods). Examples of certain of these embodiments include those described in U.S. Pat. No. 6,465,182 and published PCT application WO 99/23256; as well as published U.S. Patent Application No. 2003/0036069 and Jordan et al., Proc. Nat'l Acad. Sci. USA (Mar. 5, 2002) 99: 2942-2947.

In certain embodiments, the test and/or references sample(s) may be amplified as part of the sample generation protocol. In certain of these certain embodiments, the resultant amplified genomic sample has substantially the same complexity as the initial genomic source from which it is prepared wheras in other of these embodiments, the resultant genomic sample has reduced complexity with respect to the initial genomic source (as described).

### Detection Nucleic Acid Pairs

In certain embodiments, the methods of the present invention employ detection nucleic acid pairs in comparing the copy number of a nucleic acid locus between at least two samples. However, while the description of the methods of the invention below is drawn to comparing the copy number of a single nucleic acid region between two samples, the subject invention finds use in comparing the copy number of multiple nucleic acid regions (e.g., multiple genomic loci) between two or more samples simultaneously. As such, in the subject invention, the number of samples assayed includes two or more samples, three or more samples, about 5 or more samples, about 10 or more samples, about 50 or more samples, etc. Further, the number of loci of interest assayed by the present invention in the samples includes one or more loci, two or more loci, three or more loci, about 10 or more loci, about 30 or more loci, about 50 or more loci, etc. In general, the only limitation with regard to the number of samples and the number of loci that can be assayed simultaneously is the sensitivity of the detection system employed (described in more detail below).

Following the preparation of the genomic samples as discussed above, representative embodiments of the invention include contacting a first genomic sample (e.g., a reference sample) under specific hybridization conditions with a first pair of detection nucleic acids specific for a genomic locus and second genomic sample (e.g., a test sample) under specific hybridization conditions with a second pair of detection nucleic acids, where the first and second pair of detection nucleic acids are specific for the same genomic locus. As reviewed in greater detail below, each pair of detection nucleic acids specific for a genomic locus of interest is designed to bind (e.g., hybridize) to the genomic locus of interest at adjacent locations. The locus thus serves to position each pair of detection nucleic acids in such a manner as to allow them to be covalently linked (e.g., joined by ligation or other techniques). In order to differentiate the first and second matched pair of detection nucleic acids from one another (e.g., in the detection steps described below), the first detection pair is designed to differ in at least one physical parameter from the second detection pair when covalently joined. Non-limiting examples of physical parameters of interest include length, mass, spectral characteristic, and combinations thereof (described in more detail below). In certain embodiments, the distinguishing physical parameter is employed to allow the pairs to be physically separated.

As noted above, certain embodiments to the invention include analyzing a third genomic sample with a third detection pair, where the third detection pair is distinguishable from the first and second when covalently joined. Further, in certain embodiments, detection pairs specific for a second (or third, or fourth, etc.) genomic locus may be added to the first and second samples, providing that each detection pair incorporates a distinguishing physical parameter that imparts a detectible difference from each of the other detection pairs employed in the assay. In this way, a detected physical parameter can be associated with a particular sample and locus being compared.

Representative matched pairs of detection nucleic acids are provided for comparing copy number at a defined genetic locus. As noted above, each nucleic acid of the detection pair is designed to bind specifically to a genomic locus of interest under the binding conditions of the assay (e.g., stringent hybridization conditions). The subject detection nucleic acids are a polymer of nucleotides, where the polymer can be a variety of lengths, e.g., including from about 5 to about 1,000 bases, such as from about 20 to about 500 bases, and including from about 30 to about 100 bases. The subject detection nucleic acids may contain one or more types of nucleotides (e.g., deoxyribonucleotides or ribonucleotides) or other compounds such as peptide nucleic acids (PNA) as described in U.S. Pat. No. 5,948,902 and the references cited therein, produced either synthetically or in vivo, which can hybridize with naturally occurring nucleic acids in a sequence specific manner analogous to that of two naturally occurring nucleic acids, e.g., can participate in Watson-Crick base pairing interactions. The subject detection nucleic acids are specific for a target genomic sequence, i.e. a genomic sequence of interest. By target genomic locus is meant a genomic location or region of interest to be assayed. By target genomic sequence is meant the specific genetic sequence to which the instant detection nucleic acids are designed to hybridize. The target genomic sequence may be within the target genomic locus or it may be genetically linked to the same, as discussed in greater detail below.

The target genomic sequence can be any sequence in a genome of interest. In certain embodiments, the target genomic sequence is a gene, i.e. an expressed sequence which is transcribed to mRNA, or any sequence which regulates the expression of that mRNA. In certain embodiments the target genomic sequence is a region which is not itself transcribed to RNA but which is genetically linked to the same, i.e. finds a high probability of cosegregating with a gene during homologous recombination. In certain other embodiments, the target genomic sequence is a structural element or intergenic sequence, by which is meant a sequence not directly associated with any known transcribed sequence.

The target sequence may be of any size. In embodiments in which the detection nucleic acids are synthetic, the size of the target sequence is limited solely by the capacity to synthesize a matched pair of detection nucleic acids of sufficient length to bind specifically to it (e.g., hybridize to it under conditions of the assay). In certain embodiments, the length of the target genomic sequence is from about 5 to about 5,000 nucleotides, such as from about 10 to about 1,000 nucleotides, from about 50 to about 500, and including from about 60 to about 300 nucleotides in length.

As reviewed in greater detail below, certain embodiments of the methods of the invention exploit the capacity of ligating agents to covalently join two nucleic acids which are adjacently hybridized to a target sequence. As such, a subject matched pair of detection nucleic acids have sequences complementary to flanking regions of a genetic locus, which is to say that the binding specificities of the matched pair of detection nucleic acids enable their hybridization to adjacent contiguous sites at the genetic locus, such that the pair bind to sequences which are nonoverlapping and directly adjacent to one another, with the 3′-most base of the first target sequence directly preceding the 5′-most base of the second target sequence found in the genomic source. As such, the matched pair of detection nucleic acids have sequences complementary to flanking regions of the genetic locus of interest.

Representative embodiments of the methods of the invention exploit the capacity of physical separation protocols known to the art to physically separate nucleic acids of differing length and/or mass. As such, for each locus and/or sample being interrogated, a number of subject matched pairs of detection nucleic acids is synthesized which corresponds to the number of biological samples to be assessed, all of which pairs can hybridize to that locus. A representative pair of detection nucleic acids consists of a 5′ and a 3′ nucleic acid which hybridize to flanking regions of the locus as described above, where 5′ and 3′ refer generally to the orientation of binding of each nucleic acid as is commonly used in the art.

For each genetic locus to be tested, at least a first and a second pair of detection nucleic acids is used, which in many embodiments correspond to test and reference samples. At least one of the 5′ and 3′ detection nucleic acids of the first and second pairs differ from one another by at least one detectable physical parameter (e.g., mass, length, etc.).

For example, a set of detection pairs for a locus may be designed such that the first and second detection pair contain the same 5′ nucleic acid but contain distinguishable 3′ nucleic acids, e.g., the 3′ nucleic acid of the first detection pair is 30 bases long whereas the 3′ nucleic acid of the second detection pair is 40 bases long. In this exemplary embodiment, the 10 extra bases in the 3′ nucleic acid of the second detection pair can be part of the region of complementarity between the 3′ detection nucleic acid of the second pair and the genomic sequence or, alternatively, the 10 extra bases can be non-specific (i.e., non-hybridizing) flanking sequence. The use of such non-hybridizing flanking sequences in the design of detection pairs allows for a high degree of flexibility in assaying multiple samples, assaying multiple genomic loci (i.e., multiplex assays) or a combination of both, as the inclusion of such flanking sequences will not significantly alter hybridization to the genomic locus of interest while allowing differentiation of each of the ligated products.

Hybridization of the matched pair of detection nucleic acids at flanking sites at the target locus sets each pair in its respective sample in a position to be covalently joined by a joining agent. The joining agent covalently joins any detection nucleic acids hybridized to flanking regions of a locus in the first and second genomic samples to produce first and second covalently joined detection nucleic acids. Once covalently joined by ligation or a similar process, the joined matched pair in the first sample differs in at least one physical parameter from the joined matched pair in the second sample. Detecting the relative amounts of the first and second covalently joined detection nucleic acids provides a means to compare the copy number of the locus in first and second genomic samples. In certain embodiments, the first and second joined pairs are isolated and detected by employing a physical separation protocol, as described in further detail below.

As noted above, detectible physical parameters of interest include one or more of: the length of the nucleic acid, the mass of the nucleic acid, the spectral characteristics of the nucleic acid (e.g., the presence of a fluorescent moiety), or any other detectable characteristic that can be used to differentiate one joined pair from another.

By “length of the nucleic acid” is meant the number of biomonomers covalently joined to form a biopolymer. In certain embodiments of the invention, the lengths of at least one of the 5′ and 3′ detection nucleic acids of the first and second matched pairs of detection nucleic acids are sufficiently different to allow the physical separation of the pairs once they have been covalently joined. In such embodiments, the difference in length needed for separation of the pairs depends upon the number of samples to be measured and compared, the method(s) of physical separation being used, the respective lengths of the matched detection nucleic acids prior to covalent joining, the sensitivity of the instrument used to measure the amounts of joined nucleic acids, and other factors with which those skilled in the art will be familiar. In representative embodiments of the invention at least one of the 5′ and 3′ detection nucleic acids of the first and second matched pairs of detection nucleic acids may differ in length by from about 1 to about 1000 monomers or more, such as from about 1 to about 500 monomers, including from about 1 to 200 monomers, from about 1 to about 100, and including from about 1 to about 20 monomers. The length difference required may also be experimentally determined as desired in order to practice the disclosed method using a physical separation and/or detection technique. In certain embodiments of the invention, separation of the covalently joined pairs of detection nucleic acids is accomplished using a chromatographic protocol. Such chromatographic protocols, which may find use in the invention, are subsequently reviewed in greater detail.

In certain embodiments of the invention, the instant method includes a comparative measurement of the products of the covalent joining of detection nucleic acids which are specific for the same region but are of different lengths. In these embodiments, the sequences of the detection nucleic acids are such that those which are specific for different target sequences at the same genetic locus have substantially the same affinity for their target sequence; in other words, given the desired lengths of the detection nucleic acids, their target sequences are chosen so that all anneal to their respective target sequences under substantially the same hybridization conditions. Parameters implicated in the performance of detection nucleic acids in hybridizing to their target sequences include melting temperature, 3′-terminal stability, internal stability, and propensity of potential detection nucleic acids to form stem loops or dimers. These parameters can be assessed using any convenient method, including available software programs such as OLIGO (Molecular Biology Insights), Primer Express (PE Applied Biosystems), and Primer Premiere (Premiere Biosoft International). In certain embodiments, self-self calibration experiments are performed to characterize differences between detection nucleic acids (if they exist) which are then used to adjust accordingly.

In certain embodiments, the detectible physical parameter is imparted on the desired nucleic acid by a molecular tag. In certain embodiments, the tag is a mass tag. In these embodiments, the covalently joined pairs of detection nucleic acids used in the assay (e.g., the first and second detection pair) can be of the same length, and in such a case are distinguished by variations in mass of the detection nucleic acid. By “mass of the detection nucleic acid” is meant the chemical mass of all the atoms in the biopolymeric subject detection nucleic acid and any covalently attached moieties. In certain embodiments of the invention, the mass of at least one of the 5′ and 3′ detection nucleic acids of the first and second matched pairs of detection nucleic acids are sufficiently different such that their relative amounts can be measured (e.g., following the physical separation of the pairs once they have been covalently joined). In such embodiments, the difference in mass required for distinguishing of the pairs depends upon the number of samples to be detected and compared, the method(s) of physical separation and detection being used, the respective masses of the matched detection nucleic acids prior to covalent joining, the sensitivity of the instrument used to measure the amounts of joined nucleic acids or associated mass tags, and other factors with which those skilled in the art will be familiar.

In certain embodiments, at least one of the 5′ and 3′ detection nucleic acids in a matched pair is modified to have a different mass by the presence of a mass label (or tag) bound to one of the detection nucleic acids. In this manner, different joined pairs of detection nucleic acids which are specific for the same locus (e.g., first and second detection pairs used in a first and second genomic sample) can be distinguished. For example, the difference in mass between joined pairs of detection nucleic acids with a mass label and joined pairs of detection nucleic acids without a mass label can be detected. In addition, differences in mass between different joined pairs of detection nucleic acids where each has a mass label and the two mass labels are of differing mass can also be detected.

Any convenient method for the addition of mass labels to nucleic acids can be used in practicing the present invention. Mass labels which, without limitation, may find use in the invention include nonstandard and custom phosphoroamidites, mass-modified nucleotides, and cleavable mass tags, including photocleavable and acid-cleavable light isotope-coded affinity tags. In certain embodiments, the generation of mass-labeled nucleic acids for use in the methods of the invention is accomplished by PCR in which one (or both) of the primers comprises a mass tag (e.g., a mass-modified nucleotide, mass tag, etc.). Other convenient methods of producing mass labeled nucleic acids may also be used.

In one embodiment of the instant method, the presence and size of any cleaved mass tag is determined by mass spectrometry. Mass spectrometry includes, for example, atmospheric pressure chemical ionization mass spectrometry, electrospray ionization mass spectrometry, and matrix assisted laser desorption ionization mass spectrometry.

In certain embodiments mass tags are, by way of example, covalently attached to custom phosphoramidites which are incorporated into detection nucleic acids during synthesis, for example linked to biotinylated phosphoramidites by way of an acid-labile carbamate moiety such that they can be cleaved from isolated joined pairs of detection nucleic acids prior to quantitation by mass spectrometry. In such embodiments, the mass tags attached to instant detection nucleic acids can have any mass suitable for measurement by mass spectrometry, including from about 100 kDa to about 5,000 kDa or more. Detection nucleic acids can be synthesized with a primary amine-group at the 5′-end for subsequent coupling to esters of mass tags. Mass tags with differing molecular weights can be generated by introducing various functional groups or heavy-isotope carrier labels in a mass tag parent structure to code for individual detection nucleic acids and thus for the targeted genomic locus sequence or particular sample identity. A prototypical mass tag which finds use in the invention has in its structure a reactive labeling moiety (i.e., avidin, biotin, or an alkylator moiety) and a label (i.e. a heavy-label carrier such as C13-labeled polyalanine peptide, or a diamine with a mono- or diethylene ether backbone, or other functional group).

Mass spectrometry is capable of detecting small stable molecules with high sensitivity, at a mass resolution greater than one dalton, and the detection requires only microseconds. The mass tagging approach has been successfully used to detect multiplex single nucleotide polymorphisms, and can similarly be used to assess differences in copy number by employing the inventive method. Such mass spectrometric protocols, which find use in certain embodiments of the invention, are reviewed in greater detail below.

In certain embodiments, joined detection nucleic acids pairs employed in the invention are distinguishable by two or more physical parameters. Such embodiments find use in multiplex embodiments, including multiplex embodiments in which more than two samples are being assayed. For example, the detection pairs specific for a genomic locus of interest can be distinguished, when ligated, by mass (e.g., the detection pairs for the same genomic locus of interest contacted to each sample have a distinct mass tag), while the sets of detection pairs for each genomic locus, when ligated, can be distinguishable by length (e.g., the detection pairs specific for distinct genomic loci contacted to the same sample each have different lengths). As indicated above, the number of samples and loci that can be assayed in such multiplex embodiments is generally limited by the sensitivity of the detection and separation systems employed.

### Hybridization to Target Sequence and Ligation of Detection Nucleic Acids

As summarized above, embodiments of the invention provide matched pairs of nucleic acids which, under stringent hybridizing conditions, hybridize to a genomic locus of interest at adjacent locations, such that the locus serves to position the pair to be covalently joined by ligation or other techniques.

Standard hybridization techniques (e.g., using high stringency hybridization conditions) are employed in certain embodiments. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos. 6,335,167; 6,197,501; 5,830,645; and 5,665,549; the disclosures of which are herein incorporate by reference.

As indicated above, hybridization is carried out under suitable hybridization conditions, which may vary in stringency as desired. In certain embodiments, highly stringent hybridization conditions may be employed. The term “highly stringent hybridization conditions” as used herein refers to conditions that are compatible to produce nucleic acid binding complexes between complementary binding members, i.e., between the genetic locus to be tested and complementary detection nucleic acids in a sample. Representative high stringency assay conditions that may be employed in these embodiments are provided above. In certain embodiments, a washing step may be employed following a hybridization step.

As discussed, embodiments of the invention exploit the hybridization of matched pairs of detection nucleic acids under stringent conditions to position the same for covalent joining by an agent, forming covalently joined pairs of detection nucleic acids. Such ligation may be performed by contacting the complex consisting of a matched pair of detection nucleic acids hybridized to flanking regions of their target genomic sequence with a DNA ligase or other nucleic acid joining agent. Such ligation may be performed by any agent capable of covalently joining nucleic acids hybridized to flanking regions of a genomic sequence with specificity, which is to say that nucleic acids which are not hybridized to flanking regions of a genomic sequence are not efficiently joined by the agent. Any convenient protocol may be used for the covalent joining, including standard protocols for using DNA ligase, protocols involving the joining of moieties covalently attached to peptide nucleic acids (PNA), and others suitable for this purpose.

In certain embodiments, the joined pairs of detection nucleic acids are subjected to a PCR reaction prior to separation and detection. In certain of these embodiments, the detection nucleic acid pairs employed include unique PCR primer binding sites (e.g., PCR primer binding sites in a non-hybridizing flanking region of the detection nucleic acids, as described above). In certain embodiments, the PCR primers employed in the PCR reaction are labeled with a mass tag or include a mass-modified nucleic acid moiety.

In certain embodiments, a PCR step is employed in multiplex assays of the present invention. For example, detection nucleic acid pair sets for each loci can be designed in which PCR primer binding sites, e.g., in the form of non-hybridizing flanking sequences, are included. In one example, the PCR primer binding sites for all of the detection nucleic acids employed in the assay are identical (i.e., universal primer binding sites). In this example, the joined detection nucleic acids for the genomic loci of interest in each sample are distinguishable from each other by length (described above). After joining, both samples are subjected to a PCR reaction using the universal primers, where the universal primers for the first sample and second sample are distinguishable by mass (e.g., have distinguishable mass tags). In this way, the joined detection nucleic acids can be distinguished one from the other by both length and mass.

Other implementations of a PCR reaction in the present invention can also be employed, including those in which each different detection pair (or set of detection pairs) has distinct PCR primer binding sites. In such embodiments, a multiplex PCR reaction is performed on each sample being assayed. For each sample, PCR primer pairs specific for each detection pair are used, where each PCR primer pair produces a product having a distinct mass (e.g., each primer pair has a distinct mass tag). The number of PCR primer pairs that can be employed in such embodiments include up to about 4 or more, up to about 5 or more, up to about 10 or more, and including up to about 20 or more.

Additional embodiments that include a PCR step are readily envisioned by those of skill in the art, and as such, the description above is not meant to be limiting.

In certain embodiments, once covalently joined, the resulting mixtures may be loaded onto a physical separation device. Since the joined pairs of detection nucleic acids are distinguishable by their different lengths, mass labels, or spectral characteristics, they are mixed prior to loading onto the physical separation device for separation and detection (e.g., quantitation).

### Physical Separation of Covalently Joined Detection Nucleic Acids

In certain embodiments of the invention, the mixed joined pairs of detection nucleic acids are physically separated by employing a chromatographic protocol. In certain embodiments, the chromatographic protocol is a liquid chromatographic (LC) protocol. In embodiments where the subject joined pairs of detection nucleic acids are of different lengths, the presence of the different species is distinguished and measured by employing data obtained from the chromatographic step. In embodiments where the subject joined pairs of detection nucleic acids are of the same length and bear mass tags of differing masses, a chromatographic protocol may be used to isolate the mixture of all mass-tagged joined pairs of detection nucleic acids, which may then be subjected to a mass spectrometric (MS) protocol. The tandem use of these techniques for the analysis of nucleic acids (tandem LC-MS, or on-line LC-MS) is described, for example, at Huber et al., Mass Spectrometry Reviews (2001) 20:310-343. The techniques as they may find use in the subject invention are reviewed in greater detail below.

### Chromatography

Joined pairs of detection nucleic acids may be isolated according to the present invention using any convenient chromatographic protocol. In many embodiments of the invention, isolation will be performed using a liquid chromatographic protocol. The separation of nucleic acids of different sizes using liquid chromatography is well known to the art. Suitable methods and general techniques, including liquid chromatographic techniques suitable for use prior to mass spectrophotometric protocols, are reviewed in Huber et al., Mass Spectrometry Reviews (2001) 20:310-343. Liquid chromatography, such high performance liquid chromatography (HPLC), generally refers to a technique for partitioning a sample, or more specifically the components of a sample, between a mobile phase (typically containing an ion-pairing reagent) and a stationary phase.

Certain embodiments of the invention may employ reversed phase HPLC which is based upon solvophobic interactions between the hydrophobic nucleobases of nucleic acids and the nonpolar surface of a stationary phase, described in more detail below. In this technique, elution is achieved by the application of gradients of increasing concentration of organic solvents, for example, acetonitrile or methanol, in aqueous mobile phase. Reversed-phase HPLC is compatible with the subsequent use of eletrospray-based mass spectrometric protocols, and is effective on single-stranded oligonucleotides modified with hydrophobic groups.

Certain embodiments of the invention may employ ion-pair reversed-phase HPLC which uses a hydrophobic stationary phase and a hydroorganic mobile phase modified with an ion-pair reagent that consists of an amphiphilic charge-carrying ion with hydrophobic groups and a small hydrophilic counterion. Triethylammonium acetate is a commonly applied ion-pair reagent suitable to this purpose. Because of its high resolving capacity and mass spectrometric protocol-compatible mobile phases, ion-pair reversed-phase HPLC is commonly used prior to such protocols. Single stranded oligodeoxynucleotides exhibit sequence-dependent retention in this system however, and elution conditions for isolation of detection nucleic acids is therefore predetermined experimentally when used in the subject invention.

In certain embodiments of the present invention, a chromatographic method utilizes conditions effective to denature duplexes during sample elution to thereby enable the separation and identification of different nucleic acid molecules in a mixture. A variety of methods can be used for denaturation of nucleic acid molecules. Elevated temperatures can be used for carrying out the separation method of the invention. Such temperatures are often above 70 degrees C., depending on the specific sequence of the detection nucleic acid. Alternatively, a chemical reagent for denaturation can be used in the mobile phase. Examples of such chemical reagents include dimethylsulfoxide, urea, formamide, glycerol, and betaine.

### Stationary Phase

In certain embodiments of the invention, a test mixture containing a mixture of nucleic acid samples is applied to a stationary phase. Generally, the stationary phase is a reversed phase material (which can include a base material and a chemically bonded phase), which is hydrophobic and less polar than the starting mobile phase (i.e., the starting gradient in a gradient elution mode). A variety of commercially available reversed phase solid supports may be utilized in the present nucleic acid separation method as long as they are able to separate unlabeled nucleic acid molecules.

Reversed phase columns or column packing materials which may find use in the invention are typically composed of inorganic or organic materials, which may or may not be functionalized, such as silica, cellulose and cellulose derivatives such as carboxymethylcellulose, alumina, zirconia, polystyrene, polyacrylamide, polymethylmethacrylate, and styrene copolymers (e.g., a styrene-divinyl copolymer formed from (i) a styrene monomer such as styrene, lower alkyl substituted styrene (in which the benzene ring contains one or more lower alkyl substituents), alpha-methylstyrene and lower alkyl alpha-methylstyrene and (ii) a divinyl monomer such as C₄ -C₂₀ alkyl and aryl divinyl monomers including divinylbenzene and divinylbutadiene).

One stationary support of use in the subject methods is a wide pore silica-based alkylated support. The base material composing the solid support is typically alkylated. “Alkylated” as used in reference to the solid support refers to attachment of hydrocarbon chains to the surface of the base material of the solid support. The hydrocarbon chains may be saturated or unsaturated and may optionally contain additional functional groups attached thereto. The hydrocarbon chains may be branched or straight chain and may contain cyclic groups such as cyclopropyl, cyclopropyl-methyl, cyclobutyl, cyclopentyl, cyclopentylethyl, and cyclohexyl.

Alkylation of the base material prevents secondary interactions and can improve the loading of the stationary phase with the ion-pairing reagent to promote conversion of the solid support into a dynamic anion-exchanger. Typically, the base material is alkylated to possess alkyl groups containing at least 3 carbon atoms, generally about 3 to about 22 carbon atoms, and preferably contains about 4 to about 18 carbon atoms. The alkylated solid support phase may optionally contain functional groups for surface modification. The presence or absence of such functional groups will be dictated by the nature of the sample to be separated and other relevant operational parameters.

The stationary phase may also include beads having a particle size of about 1 micron to about 100 microns. As used herein, the particle size is determined by measuring the largest dimension of the particle (typically, the diameter for a spherical particle).

In certain embodiments, a stationary phase for use in the present method has pores with sizes ranging from less than about 30 Angstroms in diameter (e.g., nonporous materials) up to about 1000 Angstroms in size. “Nonporous stationary support” refers to a solid support composed of a packing material having surface pores of a diameter that excludes permeation of sample compounds into the pore structure, typically of less than about 30 Angstroms in diameter. In using nonporous polymeric support materials, the relatively small pore size excludes many sample compounds from permeating the pore structure and may promote increased interaction with the active surface. The stationary phase may also contain more than one type of pore or pore system, e.g., containing both micropores (less than about 50 Angstroms) and macropores (greater than about 1000 Angstroms). For achieving separations of samples containing heteroduplexes and homoduplexes of up to about 1000 base pairs in size, the stationary phase may have a surface area of about 2 m.sup.2/g to about 400 m.sup.2/g, and including about 8 m.sup.2/g to about 20 m.sup.2/g, as determined by nitrogen adsorption.

Commercially available stationary phases include a wide pore silica-based C18 material commercially available under the trade designation “ECLIPSE ds DNA” from Hewlett Packard Newport, Newport, Del., and an alkylated polystyrene-divinylbenzene nonporous material commercially available under the trade designation “DNASep” from Transgenomic, San Jose, Calif.

### Mobile Phase

The selection of aqueous mobile phase components will vary depending upon the nature of the sample and the degree of separation desired. Any of a number of mobile phase components typically utilized in ion-pairing reversed phase HPLC are suitable and may find use in the present invention. Several mobile phase parameters (e.g., pH, organic solvent, ion-pairing reagent and counterion, and elution gradient) may be varied to achieve optimal separation, such as the percent organic solvent, temperature, and concentration of the components.

Ion-pairing reagents which may find use in the invention include those which interact with ionized or ionizable groups in a sample to improve resolution including both cationic and anionic ion-pairing reagents. Cationic ion-pairing agents for use in the invention include amines such as lower alkyl primary, secondary, and tertiary amines (e.g., triethylamine (TEA)), ammonium salts such as lower trialkylammnonium salts of organic or inorganic acids (e.g., triethylammonium acetate) and lower quaternary ammonium salts such as tetrabutylanmmonium phosphate. Anionic ion-pairing agents include perfluorinated carboxylic acids. Herein, “lower alkyl” refers to an alkyl group of one to six carbon atoms, as exemplified by methyl, ethyl, n-butyl, i-butyl, t-butyl, isoamyl, n-pentyl, and isopentyl.

The hydrophobicity of the ion-pairing agent will vary depending upon the nature of the desired separation. For example, tetrabutylammonium phosphate is considered a strongly hydrophobic cation while triethylamine is a weak hydrophobic cationic ion-pairing reagent. Generally, ion-pairing agents are cationic in nature for acids and anionic for bases. One such ion-pairing agent for use in the invention is triethylammonium acetate (TEAA).

In certain embodiments of the invention, solvents for use in the mobile phase are organic solvents. The organic solvent, occasionally referred to as an organic modifier, is any organic (e.g., non-aqueous) liquid suitable for use in the chromatographic separation methods of the present invention. Generally, the organic solvent is a polar solvent (e.g., more polar than the stationary support) such as acetonitrile, methanol, ethanol, ethyl acetate, and 2-propanol. An exemplary solvent is acetonitrile.

The concentration of the mobile phase components will vary depending upon the nature of the separation to be carried out. The mobile phase composition may vary from sample to sample and during the course of the sample elution. The concentration of the ion-pairing agent in the mobile phase in certain embodiments is less than about 1.0 molar, such as within a range of about 50 mM to about 200 mM, and for example at a concentration of about 100 millimolar. The mobile phase includes less than about 40% by volume of an organic solvent in certain embodiments.

Samples are typically eluted by starting with an aqueous or mostly aqueous mobile phase containing an ion-pairing agent and progressing to a mobile phase containing increasing amounts of an organic solvent. Any of a number of gradient profiles and system components may be used to achieve the denaturing conditions of the present invention. One such exemplary gradient system is a linear binary gradient system composed of 0.1 molar triethylammonium acetate, 0.1 millimolar ethylenediaminetetraacetic acid (EDTA), and 25% acetonitrile in a solution of 0.1 molar triethylammonium acetate and 0.1 millimolar EDTA. The EDTA is typically used when the reversed phase support is a silica-based material to prevent DNA adsorbing to the silica and/or metal chelation. Under chromatographic conditions using 100 mM triethylammonium acetate, ion-pair reversed-phase HPLC with UV detection permits the separation of single-stranded oligodeoxynucleotides at single-nucleotide resolution in a size range from 3-mers to longer than 80-mers (Huber et al., LC GC Int. (1996) 14:114-127).

One way to achieve the denaturing conditions of the present invention is to modulate column temperature. The column temperature will depend upon the putative sequence (base composition) of the nucleic acid samples to be separated and the particular mass tag, if any. Thus, the choice of stationary phase, the choice of mobile phase, pH, flow rate, and the like, and in many embodiments, will be determined empirically.

### Microfluidic Chip

Certain embodiments of the invention employ a microfluidic chip platform to perform liquid chromatography. A microfluidic chip is an integrated, miniaturized device which is capable of accommodating chromatographic protocols. In a representative microfluidic chip, a capillary is packed with packing material, and the ends of the capillary are connected to a high voltage power supply. The packing material is of the type which is used in capillary electrochromatography. Sample substances which have been introduced into the capillary are moved through the capillary by means of the electric field provided by a power supply. The movement due to the electric field is dependent on the respective mobility of the sample substances. Because there is an interaction between the sample substances and the packing material, there is retention in the capillary which also is dependent on the specific sample substance. Thus, there are combined separation modes in the capillary, i.e., retention and electromigration. A first detector is arranged near the end of the packing and a second detector is arranged near the end of the capillary. The detectors may be absorbance detectors, fluorescence detectors or other detectors.

In certain embodiments of the invention, a microfluidic chip includes specialized on-chip detectors which are sensitive to the presence of a specific isolated (i.e. length-separated) joined detection nucleic acid or set of the same. The chip may thereby be designed for a specific use in order to enhance the resolution, sensitivity and multiplexing rate potential of an assay according the present method. Such an embodiment may thereby avoid any necessity to amplify a genomic source or to label a sample prior to chromatographic separation.

### Detection

In certain embodiments of the invention, unlabeled joined pairs of detection nucleic acids are detected upon elution from the chromatographic separation device by employing ultraviolet absorbance to measure the concentration of each joined pair as it emerges, as is well known in the art. Detection of absorption at 260 nm is a commonly used method for measuring the concentration of single-stranded nucleic acids in solution. In such embodiments, a mixture containing a plurality of unlabeled nucleic acid samples is applied to a first end of a chromatography column containing a stationary phase, preferably in the presence of a mobile phase. These samples are run through the column under conditions to denature the nucleic acid molecules and separate the samples. Upon elution, the nucleic acid species of each of the separately labeled nucleic acid sample pass through a detection zone in series as separated by length, each sample generating an absorption peak which is resolvable from the peaks of the other nucleic acid samples due to their asynchronous emergence from the column.

In other embodiments, the subject pairs of detection nucleic acids are labeled with a fluorescent or other label prior to use in the method as claimed. By incorporating detectable tags, a mixture containing a plurality of labeled and unlabeled nucleic acid samples can be applied to a first end of a chromatography column containing a stationary phase in the presence of a mobile phase. These samples are run through the column under conditions to denature the nucleic acid molecules and separate the samples. Upon elution, the nucleic acid species of each of the separately labeled nucleic acid sample pass through a detection zone, each sample generating a specific signal which is spectrally resolvable from the specific signals of the other nucleic acid samples. Consequently, each nucleic acid sample generates a chromatogram that can be reconstructed to represent the elution pattern of the individual nucleic acid species in this sample. This chromatogram is distinct and independent of the similarly obtained chromatograms for the other nucleic acid samples that were injected into the system.

In certain embodiments, a spectral detector is operably attached to a second end of the chromatography column. The detector can be used to detect ultraviolet absorption, or to excite the detectable tags at one wavelength and detect emissions as multiple wavelengths, or excite at multiple wavelengths and detect at one emission wavelength. Alternatively, the sample can be excited using “zero-order” excitation in which the full spectrum of light (e.g., from xenon lamp) illuminates the flow cell. Each compound can absorb at its characteristic wavelength of light and then emit maximum fluorescence. The multiple emission signals can be monitored independently. Preferably, a suitable detector can be programmed to detect more than one excitation emission wavelength substantially simultaneously, such as that commercially available under the trade designation HP1100 (G1321A), from Hewlett Packard, Wilmington, Del. Thus, the labeled nucleic acid samples eluted from the stationary phase can be detected at programmed emission wavelengths at various intervals during elution.

### Analysis

Results from the detecting or evaluating of the amount of joined pairs of detection nucleic acids may be raw results (such as peak absorbance, or fluorescence intensity readings for each feature in one or more color channels) or may be processed results. Processed results may include those obtained by subtracting a background measurement, or by rejecting a reading which is below a predetermined threshold, generating a ratio or otherwise normalizing the results, and/or forming conclusions based on the pattern read from the chromatogram (such as whether or not a particular target sequence may have been present in a greater copy number in a test sample than in a reference sample, or whether or not a pattern indicates a particular condition of an organism from which the sample came).

In certain embodiments, results are assessed by determining an amount of joined pairs of detection nucleic acids that exist as a result of hybridization with a target sequence in a genomic region. The term “amount of joined pairs of detection nucleic acids” means any assessment of amount (e.g. a quantitative or qualitative, relative or absolute assessment) by detecting signal (i.e., absorption, fluorescence) from the nucleic acid or the label associated with labeled detection nucleic acids. Since the amount of a joined pair of detection nucleic acids is proportional to the number of copies of the target sequence present during hybridization with the detection nucleic acids, an increase or decrease in copy number of the target sequence in a sample in relation to another sample can be calculated by determining the ratio of detected signals between the first and second samples. The results may be expressed using any convenient means, e.g., as a number or numerical ratio, etc. For example, the genomic copy number of a genomic region of interest in a test sample can be evaluated by comparing the amount of joined detection nucleic acids generated by hybridization with, and ligation in the presence of, the test sample relative to a reference sample with a known genomic copy number for that region. The results of the reading (whether further processed or not) may be forwarded (such as by communication) to a remote location if desired, and received there for further use (such as further processing).

### Mass Spectrometry

In certain embodiments of the invention where at least one of the first or second detection nucleic acids in a matched pair is distinguished by a mass label, mass spectrometry is employed. Following a chromatographic protocol, the mixture of joined pairs of detection nucleic acids isolated as an eluted fraction is loaded onto a mass spectrometer. Mass spectrometry is an analytical methodology used for quantitative molecular analysis of analytes in a sample. Analytes in a sample are ionized, separated according to their mass by a spectrometer and detected to produce a mass spectrum. The mass spectrum provides information about the masses and the quantities of the various analytes that make up the sample. In certain embodiments, mass spectrometry is used to determine the molecular weight or the molecular structure of an analyte in a sample and its quantity in the sample. Because mass spectrometry is fast, specific and sensitive, mass spectrometer devices have been widely used for the rapid identification, characterization and quantitation of biological analytes.

In a mass spectrometer, analyte molecules are ionized in an ion source. The masses of the resultant ions are determined in a vacuum by a mass analyzer that measures the mass/charge (m/z) ratio of the ions. When used in conjunction with a liquid chromatography device, a mass spectrometer can provide information on the molecular weight and chemical structure of compounds separated by the chromatography device, allowing identification of those components. The area under the signal intensity (i.e., radioactive decay counts or absorbance) curve, generally corresponding to maximum peak height, of a mass spectrum is quantitatively representative of the amount of analyte present in a sample.

Mass spectrometers may be configured in many different ways, but are generally distinguishable by the ionization methods employed and the ion separation methods employed. For example, in certain devices parent analyte ions are isolated, the parent ions are fragmented to produce daughter ions and the daughter ions are subjected to mass analysis. The identity and/or structure of the parent analyte ion can be deduced from the masses of the daughter ions. Such devices, generally referred to as tandem mass spectrometers (or MS/MS devices) may be coupled with a liquid chromatography system (e.g., an HPLC system or the like) and a suitable ion source (e.g. an electrospray ion source) to investigate analytes in a liquid sample.

### Analysis

Results from the detecting or evaluating of the amount of mass label attached to, or cleaved from, joined pairs of detection nucleic acids may be raw results (such as m/z ratios) or may be processed results. Processed results may include those obtained by subtracting a background measurement, or by rejecting a reading which is below a predetermined threshold, generating a ratio or otherwise normalizing the results, and/or forming conclusions based on the pattern read from the chromatogram (such as whether or not a particular target sequence may have been present in a greater copy number in a test sample than in a reference sample, or whether or not a pattern indicates a particular condition of an organism from which the sample came).

In certain embodiments, results are assessed by determining an amount of joined pairs of detection nucleic acids that exist as a result of hybridization with a target sequence in a genomic region. The term “amount of joined pairs of detection nucleic acids” means any assessment of amount (e.g. a quantitative or qualitative, relative or absolute assessment) by detecting signal (i.e., absorption, decay counts) from the mass label associated with labeled detection nucleic acids. Since the amount of a mass label coordinated to or cleaved from a joined pair of detection nucleic acids is proportional to the number of copies of the target sequence present during hybridization with the detection nucleic acids, an increase or decrease in copy number of the target sequence in a sample in relation to another sample can be calculated by determining the ratio of detected signals between the first and second samples. The results may be expressed using any convenient means, e.g., as a number or numerical ratio, etc. For example, the genomic copy number of a genomic region of interest in a test sample can be evaluated by comparing the amount of mass label coordinated to joined detection nucleic acids generated by hybridization with, and ligation in the presence of, the test sample relative to a reference sample with a known genomic copy number for that region. The results of the reading (whether further processed or not) may be forwarded (such as by communication) to a remote location if desired, and received there for further use (such as further processing).

In certain embodiments, the subject methods include a step of transmitting data from at least one of the detecting and deriving steps, as described above, to a remote location. By “remote location” it is meant a location other than the location at which the array is present and the array assay, e.g., hybridization, occurs. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

While the embodiments described above are drawn to methods in which a single test and reference sample are employed, the subject invention is not limited to such embodiments. For example, in certain embodiments, multiple test samples can be prepared and assessed relative to a single reference sample, to multiple different reference samples, or to each other. As such, the categorization of which sample is called the test sample and which is called the reference sample depends on the specific implementation of the methods disclosed herein.

The above description is merely representative of ways of performing the disclosed method and is in no way limiting.

### Utility

The subject detection nucleic acids and methods of using the same find use in a variety of different applications. One application in which the subject detection nucleic acids and methods find use is the evaluation of the copy number of one or more target genomic domains.

In such embodiments, at least a first and second genomic source are employed, of which one is a reference in which the genomic copy number of the target genomic domain is known prior to the experiment. In these representative embodiments, a matched pair of detection nucleic acids hybridize under stringent conditions to the genomic locus of interest at adjacent locations, such that the locus thereby serves to position the pair to be covalently joined. As described in detail above, at least one of each matched pair of subject detection nucleic acids is:

A) of a different length from those in other pairs, or

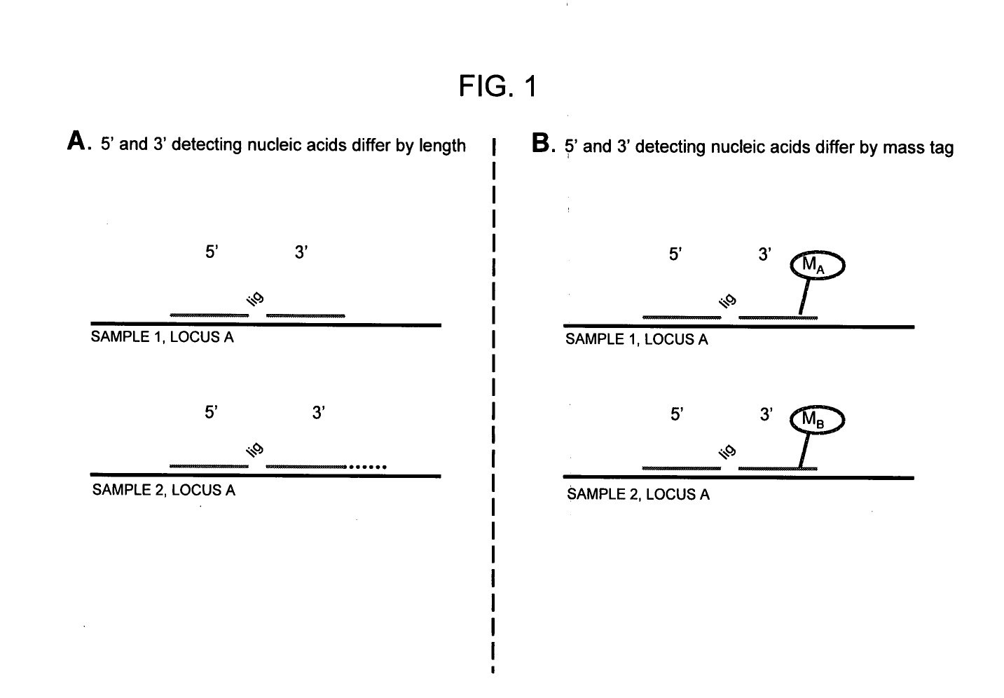

B) of the same length but physically distinguishable [e.g., coordinated, either permanently or cleavably, to a tag that distinguishes one pair from another (e.g., a mass tag, fluorescent tag, etc., as shown in FIG. 1) or distinguishable by its native mass].

Once hybridized, the mixture is contacted with an agent capable of selectively covalently joining the hybridized detection nucleic acids. In certain embodiments, the joining agent is a DNA ligase and the ligation is conducted according to standard protocols for single stranded ligation of DNA. As seen in FIG. 1, the positioning of each matched pair at flanking positions at the target sequence enables their efficient ligation. The generation of ligated matched pairs of detection nucleic acids is dependent upon the abundance of the target sequence in each sample, and is thus representative of the relative copy number in the samples. Accordingly, increases or decreases in the copy number of a test sample relative to a reference sample are determined using the disclosed method.

Following covalent joining (i.e., ligation), the resulting mixture of genomic source material, covalently joined and unjoined pairs of detection nucleic acids is then subject to a chromatographic step as discussed above. In certain embodiments, the joined pairs of detection nucleic acids will be segregated by length, detected by UV absorption, and their peak absorbencies read and compared as a ratio to determine the relative presence of the target genomic locus in the reference and test samples. For example, an increase in the absorption peak in a test sample relative to that of a reference sample is indicative of an increased copy number of the target genetic locus being assayed, etc.

In certain embodiments, the chromatographic step does not segregate the joined pairs of detection nucleic acids from one another, instead serving as a means to isolate all labeled joined pairs of detection nucleic acids from the unjoined fraction and from the genomic source material (including both single locus and multiplex loci embodiments of the invention). In such embodiments, the fraction is detected by UV absorption or fluorescence and collected. In certain embodiments, prior to mass spectrometry, the mass tags may be cleaved from the detection nucleic acids if the tags are so designed, which in some cases can aid in resolving the mass spectra and obtaining accurate data. The labeled detection nucleic acids or their cleaved tags are loaded onto a mass spectrometry device which distinguishes them by mass and allows the quantitation of each species. An increase in the detected amount of tag or tags coordinated to or cleaved from pairs of detection nucleic acids from a test sample relative to that detected for a reference sample is indicative of an increased copy number of the target genetic locus being assayed, etc.

The methods of the subject invention are applicable to evaluating the copy number of loci in genomic samples from a wide variety of cells and cell types, including genomic samples derived from both prokaryotic and eukaryotic cells.

In certain embodiments, genomic copy number is being evaluated to assess the presence or type of a genetic lesion which is not congenital, such as a neoplastic condition, i.e., cancer. In such embodiments a reference composition is also assayed. In such cases the reference composition may be from the same organism that was used to generate the test composition but where the sample used is known not to contain the lesion. Accordingly, in these embodiments, a genomic sample is prepared and used to make at least one test target composition and at least one reference target composition from equal cell number aliquots of the genomic sample. In certain of these embodiments, the reference target composition differs from the test target composition in that it is not associated with a tumor or other neoplasia.

In still other embodiments, where genomic copy number is evaluated to test for the presence of a congenital genetic lesion such as Trisomy 21, the reference target composition differs from the test target composition in that it is obtained from a different subject in which the copy number of the locus or loci to be assessed is known prior to the experiment.

Accordingly, such embodiments of the present invention may be used in methods of comparing abnormal nucleic acid copy number and mapping of chromosomal abnormalities associated with disease. In certain embodiments, the subject methods are employed in applications that use samples obtained from subjects to undergo diagnosis for such conditions. Analysis of processed results of the described experiments provides information about the relative copy number of nucleic acid domains, e.g. genes, in genomes.

Such applications compare the copy numbers of sequences capable of binding to the detection nucleic acids. Variations in copy number detectable by the methods of the invention may arise in different ways. For example, copy number may be altered as a result of amplification or deletion of a chromosomal region, e.g. as commonly occurs in cancer.

The subject methods are suitable for simultaneous assessment of the copy number of a large number of genomic regions. The disclosed methods permits joined pairs of detection nucleic acids, used to assay different samples and different loci, to be loaded in parallel onto a physical separation device or devices and distinguished from one another. Accordingly, the number of samples and/or loci which can be assayed in multiplex is limited solely by the available lengths of potential target sequences, available mass tags and capacity of the physical separation devices to distinguish the same. In certain embodiments, a microfluidic chip is used to improve multiplexing potential by increasing the speed of chromatographic separation and measurement as well as by reducing the required sample amount due its capacity to accommodate specialized sensor components.

The above-described methods find use in any application in which one wishes to compare the copy number of nucleic acid sequences found in two or more populations. One type of representative application in which the subject methods find use is the quantitative comparison of copy number of one nucleic acid sequence in a first collection of nucleic acid molecules relative to the copy number of the same sequence in a second collection. The subject methods find use in the detection of both heterozygous and hemizygous deletions of sequences, as well as amplification of sequences and variation in copy number which may be characteristic of certain conditions, e.g., disease conditions. Non-limiting examples of conditions that can be detected include cancers and developmental disorders (e.g., for prenatal diagnostics).

The above descriptions are provided so that one of skill in the art may understand how the present invention may be used, and are not intended to be limiting. The subject detection nucleic acids and methods for using the same may also be employed in other applications.

### Kits

Also provided by the subject invention are kits for practicing the subject methods, as described above. The subject kits include at least a first and second pair of detection nucleic acids, including 5′ and 3′ detection nucleic acids which have sequences complementary to flanking regions of a genomic locus of interest to the user of the kit, and where at least one of the 5′ and 3′ detection nucleic acids of the first and second pairs differ from each other by at least one physical parameter. As discussed in detail above, non-limiting examples of detectible physical parameters that find use in the invention include nucleitide length, mass, and spectral characteristic (e.g. fluorescence).I.

The described kits may additionally contain third and fourth pairs of detection nucleic acids made up of 5′ and 3′ detection nucleic acids that flank a second genomic locus, or flank the same genomic locus but accommodate the assay of a larger number of samples, or may contain any additional number of pairs of detection nucleic acids which may be useful for either or both of the described purposes.

Other optional components of the kit include: a joining agent such as a DNA ligase and buffers for use with the same, and a microfluidic chip capable of detecting and measuring joined subject detection nucleic acids. The various components of the kit may be present in separate containers or certain compatible components may be precombined into a single container, as desired.

In addition to the above-mentioned components, the subject kits may further include instructions for using the components of the kit to practice the subject methods (i.e., using the detection nucleic acids in a method to evaluate the copy number of a genomic region of interest). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject nucleic acids, optional components and instructions, the kits may also include one or more control analyte mixtures, e.g., two or more control compositions for use in testing the kit.

The subject invention finds use in methods for detecting differences in genome copy number between two biological samples and, accordingly, finds particular use as a diagnostic and research tool for investigating a number of disease conditions as well as for assaying other cellular processes impacted by genome copy number variations, and as such, represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of comparing the copy number of a locus in first and second genomic samples, said method comprising:
(a) respectively contacting said first and second genomic samples under specific hybridization conditions with first and second pairs of detection nucleic acids, wherein said first and second pairs of detection nucleic acids each comprise:
(i) 5′ and 3′ detection nucleic acids having sequences complementary to flanking regions of said locus; and
(ii) at least one of said 5′ and 3′ detection nucleic acids of said first and second pairs differ from each other by at least one physical parameter;
(b) covalently joining any detection nucleic acids hybridized to flanking regions of said locus in said first and second genomic samples to produce first and second covalently joined detection nucleic acids; and
(c) detecting said first and second covalently joined detection nucleic acids to compare the copy number of said locus in said first and second genomic samples.

2. The method according to claim 1, wherein said physical parameter is length.

3. The method according to claim 1, wherein said physical parameter is mass.

4. The method according to claim 3, wherein said detection nucleic acids of differing mass differ from each other by the presence of mass labels of differing mass bound to one of said detection nucleic acids.

5. The method according to claim 1, wherein said method further comprises comparing the copy number of a second loci using third and fourth pairs of detection nucleic acids.

6. The method according to claim 1, wherein said first sample is a test sample and second sample is a reference sample.

7. The method according to claim 1, wherein said method is a method of detecting the presence of a genetic lesion.

8. The method according to claim 7, wherein said genetic lesion is associated with the presence of a disease condition.

9. The method according to claim 8, wherein said disease condition is a neoplastic disease condition.

10. The method according to claim 9, wherein said neoplastic disease condition is cancer.

11. The method according to claim 1, wherein said covalently joining comprises ligating said 5′ and 3′ detection nucleic acids.

12. The method according to claim 11, wherein said ligating is mediated by a DNA ligase.

13. The method according to claim 1, wherein said detecting employs a physical separation protocol.

14. The method according to claim 13, wherein said physical separation protocol is a chromatographic protocol.

15. The method according to claim 14, wherein said chromatographic protocol is a liquid chromatographic protocol.

16. The method according to claim 13, wherein said physical separation protocol is a mass spectrometry protocol.

17. The method according to claim 1, wherein said method is performed in a microfluidic chip.

18. A kit comprising:
(a) a first pair of detection nucleic acids comprising 5′ and 3′ detection nucleic acids having sequences complementary to flanking regions of a genomic locus; and
(b) a second pair of detection nucleic acids comprising 5′ and 3′ detection nucleic acids having sequences complementary to said flanking regions of said genomic locus;
wherein at least one of said 5′ and 3′ detection nucleic acids of said first and second pairs differ from each other by at least one physical parameter.

19. The kit according to claim 18, wherein said physical parameter is length.

20. The kit according to claim 18, wherein said physical parameter is mass.

21. The kit according to claim 20, wherein said detection nucleic acids of differing mass differ from each other by the presence of mass labels of differing mass bound to one of said detection nucleic acids.

22. The kit according to claim 18, wherein said kit further comprises third and fourth pairs of detection nucleic acids made up of 5′ and 3′ detection nucleic acids that flank a second genomic locus.

23. The kit according to claim 18, wherein said kit further comprises a ligase.

24. The kit according to claim 18, wherein said kit further comprises a microfluidic chip.
